(19) United States
(12) Patent Application Publication
(10) Pub. No.: US 2011/0287452 A1
(43) Pub. Date: Nov. 24, 2011

(54) Test Kit for Detecting Klebsiella Pneumoniae Serotype K1 and Method Using Same

(75) Inventors: Feng-Yee Chang, Taipei (TW); Chang-Phone Fung, Taipei (TW); Leung-Kei Siu, Taipei (TW); Yung-Chung Lin, Taipei (TW); Kuo-Ming Yeh, Taipei (TW); Te-Li Chen, Taipei (TW)

(73) Assignee: NATIONAL DEFENSE MEDICAL CENTER, Taipei (TW)

(21) Appl. No.: 12/827,286

(22) Filed: Jun. 30, 2010

(30) Foreign Application Priority Data
May 18, 2010 (TW) 099115840

(51) Int. Cl. G01N 33/53

(52) U.S. Cl. 435/7.32

(57) ABSTRACT

Disclosed are a test kit and method for sensitively and rapidly detecting Klebsiella pneumoniae serotype K1. By using immunochromatographic test, the test kit can sensitively, rapidly and specifically identify whether specimens contain Klebsiella pneumoniae serotype K1. The sensitivity of the test kit preferably attains 1.4×10⁵ cfu/50γ.

### FIELD OF THE INVENTION

The present invention relates to a test kit, particularly relates to a test kit for sensitively and rapidly detecting Klebsiella pneumoniae serotype K1.

### BACKGROUND OF THE INVENTION

Klebsiella pneumoniae is a Gram-negative, rod shaped bacterium found in the normal flora of the mouth, skin, and intestines. In recent years, a new type of invasive Klebsiella pneumoniae infection has raised to be a global developing disease. The capsular polysaccharides of Klebsiella pneumoniae can be classified into 77 serotypes. In western countries, K2 and K21 are the most popular serotypes. On the contrary, K1 and K2 are the popular serotypes in Taiwan. Clinically, bacteremia, liver abscess, pneumonia, urinary tract infection are the common diseases induced by Klebsiella pneumoniae serotypes K1 and K2. Conventionally, capsular serotypes are determined by capsular swelling test or counter current immunoelectrophoresis. However, drawbacks of the conventional methods include consumption of time, materials and manpower, and false-positive/false-negative results caused by cross reactions. Additionally, polymerase chain reaction (PCR) also can be used to determine Klebsiella pneumoniae serotypes. Nevertheless, PCR analysis requires longer time for preparation, expensive equipment and skilled technicians for operation.

To solve the problems in the field of Klebsiella pneumoniae detection, the applicant apply a Taiwan patent in 2007 (pub NO: 200907064, hereinafter referred to as “case A”). Case A relates to a reagent for detecting Klebsiella pneumoniae serotype K1/K2 and the method thereof. However, the reagent disclosed in case A is easier to be influenced by non-K1 strains and results the false-positive reaction, and it has low sensitivity. Therefore, the present invention provides a test kit for detecting Klebsiella pneumoniae serotype K1, which can sensitively and rapidly detect Klebsiella pneumoniae serotype K1.

Based on the drawbacks of conventional detection methods, the applicant deliberate to improve the conventional ways, and finally succeed to develop the test kit for sensitively and rapidly detecting Klebsiella pneumoniae serotype K1. Through the present invention, clinicians and researchers can quickly and correctly obtain the results, so as to be beneficial in clinical therapy and research.

### SUMMARY OF THE INVENTION

The objective of the present invention is to provide a test paper for sensitively and rapidly detecting Klebsiella pneumoniae serotype K1.

In accordance with one aspect of the present invention, the test paper comprises a matrix having an interpretation region immobilized with an antibody against Klebsiella pneumoniae serotype K1, wherein the antibody titer is determined by immunoenzymatic assay and has an OD450 difference higher than 0.55 between Klebsiella pneumoniae serotype K1 antigens and non-Klebsiella pneumoniae serotype K1 antigens.

Preferably, the matrix is a nitrocellulose membrane.

Preferably, the antibody against Klebsiella pneumoniae serotype K1 is rabbit-anti-K1 IgG.

Preferably, the antibody titer is ranged from 1:16,000 to 1:128,000.

Preferably, the antibody titer is 1:64,000.

Preferably, the test paper further comprises an immunoconjugation pad partly attached to the interpretation region.

Preferably, the immunoconjugation pad is coated with an antibody against Klebsiella pneumoniae serotype K1 and colloidal gold.

Preferably, the colloidal gold is a marker.

Preferably, the immunoconjugation pad is made of glass fiber.

Preferably, the test paper further comprises a specimen pad partly attached to the immunoconjugation pad.

Preferably, the test paper further comprises a water-absorption pad partly attached to the interpretation region.

Another objective of the present invention is to provide a test kit for sensitively and rapidly detecting Klebsiella pneumoniae serotype K1.

In accordance with another aspect of the present invention, the test kit comprises the members including a water-absorption pad, interpretation region, immunoconjugation pad and specimen pad. These members are installed on a solid substrate.

Preferably, the solid substrate is a back plate, paper plate or plastic plate.

Yet another objective of the present invention is to provide a method for sensitively and rapidly detecting Klebsiella pneumoniae serotype K1.

In accordance with yet another aspect of the present invention, the method comprises the steps of: (a) providing an isolated specimen; (b) adding the specimen to the test kit as mentioned above; and (c) observing whether an aggregation of the antibody against Klebsiella pneumoniae serotype K1 and the specimen is present.

Preferably, the specimen contains Klebsiella pneumoniae serotype K1, and thus a signal visible to the naked eye is shown due to the aggregation occurs around the antibody against Klebsiella pneumoniae serotype K1.

Preferably, the test kit used in the method further comprises a control signal represents that the test kit functions normally.

Preferably, the specimen is selected from the group consisting of bacterial culture suspension, bacterial colonies, liver abscess specimens, cerebrospinal fluid, urine, sputum, ascites and pleural fluid.

### BRIEF DESCRIPTION OF THE DRAWINGS

The above and other objects, features and other advantages of the present invention will be more clearly understood from the following detailed description taken in conjunction with the accompanying drawings in which:

FIG. 1 exhibits the antibody titer assay of the test paper;

FIG. 2 exhibits the test kit for detecting Klebsiella pneumoniae serotype K1;

FIG. 3 exhibits the plastic case of the test kit for detecting Klebsiella pneumoniae serotype K1; and

FIG. 4 exhibits the results displayed from the windows for interpretation regions of test kits, results of 1-A to 1-D represent positive reaction, negative reaction, invalid reaction and invalid reaction, respectively.

### DETAILED DESCRIPTION

### Example 1

### Preparation of Anti-Klebsiella pneumoniae Serotype K1 Serum

### 1-1. Verifying Antigen Purity and Concentration

Klebsiella pneumoniae serotype K1 was cultured with conventional medium for overnight. After overnight culture, the bacteria were killed by hot water at 70° C., so as to maintain the complete bacterial structures. To determine if the protein purity higher than 90%, SDS-PAGE was performed and thus the total amount of protein was calculated for required concentration (2 mg) of immunization.

### 1-2. Animal Immunization

3-5 ml of pre-immune serum was collected from a New Zealand rabbit. The obtained antigen as described in 1-1 was mixed with the Complete Adjuvant, and then the mixture was injected into the animal by hypodermic or splenic injection, and which is the first immunization. The serial immunizations were performed every 2 weeks, and the used adjuvant was replaced with Incomplete Adjuvant. After the fourth immunization was completed, blood sample was collected to perform western blot. And the antibody test was performed to verify that the prepared serum can be used to identify Klebsiella pneumoniae serotype K1.

### 1-3. Titer Assay

To perform the titer assay, the antibody concentration must be diluted to 10⁵ folds, and the OD280 value must be greater than 1.0 in spectrophotometer assay. If the requirement was not meet, the extended boost needed to be implemented to raise the titer. While the antibody titer meet the requirement, the total bleeding was then performed.

### 1-4. Total Bleeding

The total bleeding was performed in the sacrificed animal (heart puncture). The blood sample was centrifuged to obtain the serum.

### 1-5. Antibody Affinity Purification

The column was provided to perform the antibody affinity purification. Appropriate amount of Protein A-Sepharose (GE healthcare, 20% Ethanol contained) was loaded into the column and confirmed that no bubble exists therein. The column was washed by three times of column volume of washing buffer, and then the equal volume of binding buffer was added to wash out the washing buffer. The serum was filtered and then added into the column. Sepharose A bound to the Fc region of antibodies, thus the antibodies were kept inside the column. The unbound proteins of serum were washed out by binding buffer. The bound antibodies were eluted by elution buffer (pH 2.8-3.1). These antibodies were collected and determined the concentration by measuring the OD280 value. Tris buffer (pH 9.0) was added to neutralize the antibodies (Rabbit anti-K1 IgG) to pH 7.0-8.0 for preserving. Dialysis was performed in PBS (4° C.), and the concentration of Rabbit anti-K1 IgG was determined by spectrophotometer (OD280).

### 1-5. Optimization of Antibody Titer

According to the OD450 obtained from ELISA, the spectrophotometric difference of Klebsiella pneumoniae serotypes K1 and Klebsiella pneumoniae serotypes K2 was calculated to determine the optimum antibody titer for the test paper. Klebsiella pneumoniae serotypes K1 and K2 were added to the ELISA plate and incubated for 18 hours. The bacterial suspension was removed, and PBS was added to wash 3 times. Blocking buffer was added for 1 hr incubation (37° C.). The blocking buffer was then removed, and the various dilutions of anti-K1 antibody were added and incubated at room temperature for 2 hours. The anti-K1 antibody was removed and the PBST was added to wash 3 times. The secondary antibody was added for 2 hr incubation at room temperature. The secondary antibody was removed, and the PBST was added to wash 3 times. The TMB was then added to generate the light signals and the reaction was terminated by adding 1N HCl. The spectrophotometric difference of Klebsiella pneumoniae serotypes K1 and Klebsiella pneumoniae serotypes K2 was then calculated based on the obtained OD450.

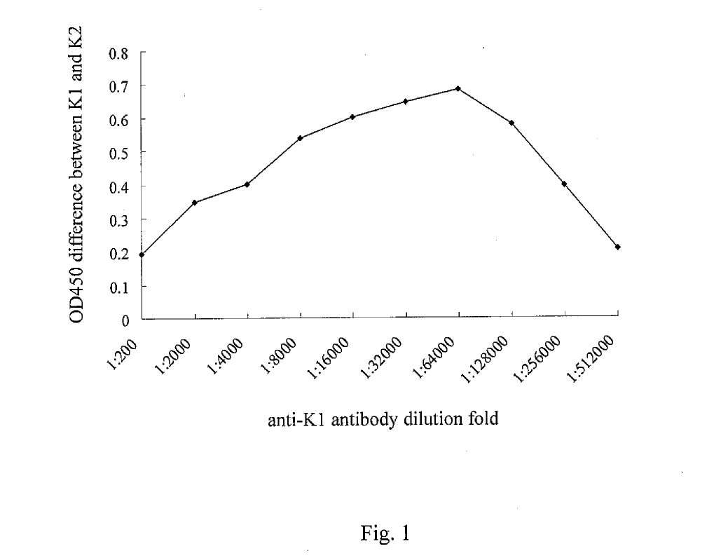

With reference to table 1, the table exhibits the optimum antibody titer of the test paper. Based on the results, as the spectrophotometric difference between Klebsiella pneumoniae serotypes K1 and Klebsiella pneumoniae serotypes K2 is higher than 0.55, the anti-K1 antibody can specifically identify the K1 antigen. Preferably, the antibody titer is ranged from 1:16,000 to 1:128,000. In particular, 1:64,000 is the optimum antibody titer, as shown in FIG. 1.

TABLE 1
Results of the optimum antibody titer of the test paper

| dilution fold of | antigen | antigen | difference of | difference of K1 |
|---|---|---|---|---|
| anti-K1 antibody | K1 | K2 | K1and K2 | and K2 >0.55 |
| 1:200 | 0.977 | 0.785 | 0.192 | − |
| 1:2000 | 0.9545 | 0.607 | 0.3475 | − |
| 1:4000 | 0.934 | 0.5315 | 0.4025 | − |
| 1:8000 | 0.962 | 0.4245 | 0.5375 | − |
| 1:16000 | 0.9035 | 0.301 | 0.6025 | + |
| 1:32000 | 0.854 | 0.206 | 0.648 | + |
| 1:64000 | 0.8005 | 0.116 | 0.6845 | + |
| 1:128000 | 0.653 | 0.0705 | 0.5825 | + |
| 1:256000 | 0.4335 | 0.0375 | 0.396 | − |
| 1:512000 | 0.2235 | 0.018 | 0.2055 | − |

### Example 2

### Fabrication of Test Kit for Detecting Klebsiella pneumoniae Serotype K1

### 1-1. Fabrication of the Interpretation Region of Test Kit

The purified antiserum (Rabbit anti-K1 IgG) as described in example 1 was diluted with the ideal antibody titer (1:64,000), and immobilized on the nitrocellulose membrane (NC membrane, >30 cm) surface by a printing device. The NC membrane was dried by constant temperature and humidity facilities for 24 hours and carried out the blocking process to form the interpretation region 12 of test kit 100. The interpretation region 12, was also called T region, can be used to detect Klebsiella pneumoniae serotype K1. On the other hand, a control (C region) was designed to monitor whether the test kit functioned normally.

### 1-2. Fabrication of Immunoconjugation Pad of Test Kit

Proper amount of antiserum (Rabbit anti-K1 IgG) and colloidal gold (25 nm) were conjugated and concentrated. The prepared antibody (1:64,000) was immobilized on the colloidal pad made of glass fiber by the printing device (flow rate=3.0 μl/cm). The pad was dried at 37° C. for 30 min to form the immunoconjugation pad 13.

### 1-3. Assemblage of Members of Test Kit

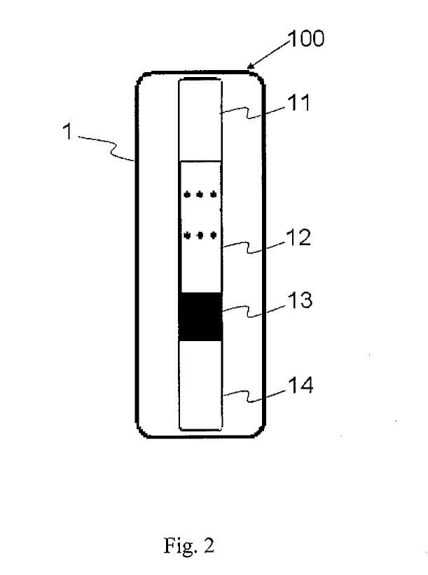

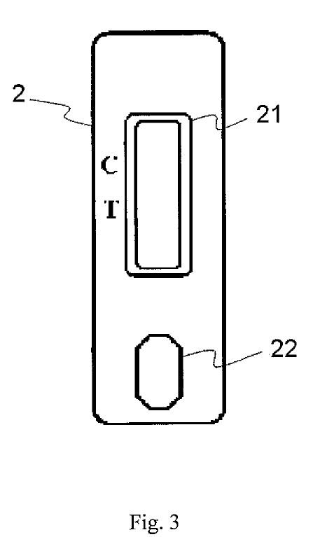

The NC membrane was removed its protection film, then pasted on the surface of solid substrate 1 and pressed by finger to verify that the NC membrane was tightly attached thereon. The water-absorption pad 11 was removed its protection film, then pasted on the surface of solid substrate 1, and had the upper edge of water-absorption pad 11 align the upper edge of the upper edge of solid substrate 1. The water-absorption pad 11 was also pressed by fingers to verify the water-absorption pad 11 is tightly attached on the solid substrate 1. The immunoconjugation pad 13 with fit size was removed its protection film, then aligned the lower edge of solid substrate 1 and pasted thereon. After verifying the specimen pad 14 overlap the immunoconjugation pad 13 in 5±2 mm, the specimen pad 14 was pressed by finger. With reference to FIG. 2, the water-absorption pad 11, interpretation region 12, immunoconjugation pad 13 and specimen pad 14 were attached on the plastic plate of the solid substrate 1 to form a test kit for detecting Klebsiella pneumoniae serotype K1. As shown in FIG. 3, the test kit further includes a plastic case 2 having a window 21 and a window 22 for inspecting the interpretation region 12 and specimen pad 14, respectively.

### Example 3

### Examination of the Test Kit for Detecting Klebsiella pneumoniae Serotype K1

### 1-1. Preparation of Specimen

Bacterial culture suspension or liver abscess specimen was stirred in saline solution to be the tested specimen. If tested specimen was cerebrospinal fluid, urine, sputum, ascites or pleural fluid, then the dilution was unnecessary. If tested specimens were bacterial colonies of Klebsiella pneumoniae serotype K1, 2-3 Colonies were picked and mixed with saline solution.

### 1-2. Examination Procedures

One drop of tested specimen was extracted by a pipette and added to the window 22 of test kit 100. After 1-3 min, whether the aggregation of antibody and specimen was occurred can be inspected through window 21, and thus the result can be determined. Compared with the prior art, the test kit of present invention exhibits that the detection process was fast, and the detection process could be finished in 5 min to avoid the false positive results.

1-3. The Results Represented from the Window

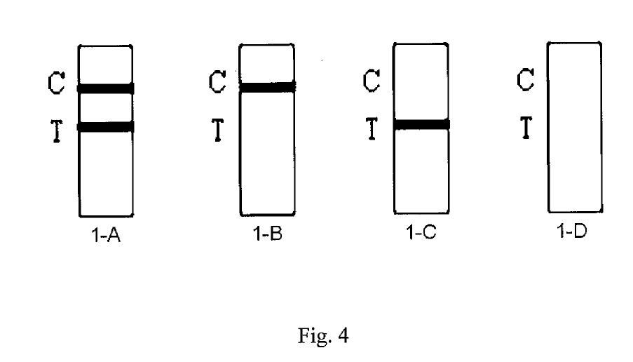

The rules of interpretation of results of the test kit in 5 min (as shown in FIG. 4) as follow:

(1) If there was no lines displayed in window 21, it was suggested that the test kit functions abnormally, and the result was thus invalid.

(2) If there function only one line displayed in T region of window 21, it function suggested that the test kit functioned abnormally, and the result was also invalid.

(3) If there was only one line displayed in C region of window 21, it was suggested that the test kit functioned normally. The result represented a negative reaction and mean that the tested specimen did not contain Klebsiella pneumoniae serotype K1.

(4) If there were two lines displayed in window 21, one appeared in C region, and another appeared in T region. It was suggested that test kit functioned normally. The result represented a positive reaction and mean that the tested specimen contained Klebsiella pneumoniae serotype K1.

### 1-4. The Minimum Bacterial Numbers of K1 can be Detected by the Test Kit

A single colony of K1 bacteria was picked and cultured in BHI medium in shaking incubator at 37° C. When the OD600=9, the bacterial suspension was serially diluted to various concentration. The bacterial suspension was added to the K1 strip for detection, and the parts of each bacterial suspension were cultured in medium plate to calculate the original bacterial concentration. According the results, the minimum bacterial numbers of K1 can be detected by the test kit was 1.4×10⁵ cfu/50γ. It is suggested that the test kit of the present invention has terrific sensitivity.

TABLE 2
Detection results by the test kit for bacterial
suspension in different concentrations

| bacterial numbers | 1.4 × 10⁷ | 10⁷ | 10⁶ | 10⁵ | 10⁴ | 10³ |
|---|---|---|---|---|---|---|
| results | + | + | + | + | − | − |

1-5. Test for 77 Klebsiella pneumoniae Capsular Serotypes

Total 77 Klebsiella pneumoniae capsular serotypes were examined by the test kit of the present invention. As shown in Table. 3, only Klebsiella pneumoniae serotype K1 was detected as positive. It is demonstrated that the test kit of the present invention has excellent specificity.

TABLE 3
Detection results of 77 Klebsiella pneumoniae capsular serotypes

| serotype | results |
|---|---|
| K1 | + |
| K2 | − |
| K3 | − |
| K4 | − |
| K5 | − |
| K6 | − |
| K7 | − |
| K8 | − |
| K9 | − |
| K10 | − |
| K11 | − |
| K12 | − |
| K13 | − |
| K14 | − |
| K15 | − |
| K16 | − |
| K17 | − |
| K18 | − |
| K19 | − |
| K20 | − |
| K21 | − |
| K22 | − |
| K23 | − |
| K24 | − |
| K25 | − |
| K26 | − |
| K27 | − |
| K28 | − |
| K29 | − |
| K30 | − |
| K31 | − |
| K32 | − |
| K33 | − |
| K34 | − |
| K35 | − |
| K36 | − |
| K37 | − |
| K38 | − |
| K39 | − |
| K40 | − |
| K41 | − |
| K42 | − |
| K43 | − |
| K44 | − |
| K45 | − |
| K46 | − |
| K47 | − |
| K48 | − |
| K49 | − |
| K50 | − |
| K51 | − |
| K52 | − |
| K53 | − |
| K54 | − |
| K55 | − |
| K56 | − |
| K57 | − |
| K58 | − |
| K59 | − |
| K60 | − |
| K61 | − |
| K62 | − |
| K63 | − |
| K64 | − |
| K65 | − |
| K66 | − |
| K67 | − |
| K68 | − |
| K69 | − |
| K70 | − |
| K71 | − |
| K72 | − |
| K74 | − |
| K79 | − |
| K80 | − |
| K81 | − |
| K82 | − |

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A test paper for detecting Klebsiella pneumoniae serotype K1, comprising a matrix having an interpretation region immobilized with an antibody against Klebsiella pneumoniae serotype K1, wherein the antibody titer is determined by immunoenzymatic assay and has an OD450 difference higher than 0.55 between Klebsiella pneumoniae serotype K1 antigens and non-Klebsiella pneumoniae serotype K1 antigens.

2. The test paper as claimed in claim 1, wherein the matrix is a nitrocellulose membrane.

3. The test paper as claimed in claim 1, wherein the antibody titer is ranged from 1:16,000 to 1:128,000.

4. The test paper as claimed in claim 1, wherein the antibody titer is 1:64,000.

5. The test paper as claimed in claim 1 further comprising a immunoconjugation pad partly attached to the interpretation region.

6. The test paper as claimed in claim 5, wherein the immunoconjugation pad is coated with an antibody against Klebsiella pneumoniae serotype K1 and colloidal gold.

7. The test paper as claimed in claim 5, wherein the immunoconjugation pad is made of glass fiber.

8. The test paper as claimed in claim 1 further comprising a specimen pad partly attached to the immunoconjugation pad.

9. The test paper as claimed in claim 1 further comprising a water-absorption pad partly attached to the interpretation region.

10. A test kit for detecting Klebsiella pneumoniae serotype K1, comprising the members of claims 1, 5, 8 and 9, wherein the members are installed on a solid substrate.

11. The test kit as claimed in claim 10, wherein the solid substrate is a back plate, paper plate or plastic plate.

12. A method for detecting Klebsiella pneumoniae serotype K1, comprising the steps of
(a) providing an isolated specimen;
(b) adding the specimen to the test kit as claimed in claim 10;
(c) observing whether a aggregation of the antibody against Klebsiella pneumoniae serotype K1 and the specimen is present.

13. The method as claimed in claim 12, wherein the specimen contains Klebsiella pneumoniae serotype K1, and thus a signal visible to the naked eye is shown due to the aggregation occurs around the antibody against Klebsiella pneumoniae serotype K1.

14. The method as claimed in claim 12, wherein the specimen is selected from the group consisting of bacterial culture suspension, bacterial colonies, liver abscess specimens, cerebrospinal fluid, urine, sputum, ascites and pleural fluid.
